(19) United States
(12) Patent Application Publication
(10) Pub. No.: US 2004/0235990 A1
(43) Pub. Date: Nov. 25, 2004

(54) 3-4-Alkylenedioxythiophene diols, their preparation and use in capacitors

(76) Inventors: Helmut-Werner Heuer, Krefeld (DE); Rolf Wehrmann, Krefeld (DE)

Correspondence Address: BAYER MATERIAL SCIENCE LLC, 100 BAYER ROAD, PITTSBURGH, PA, 15205, US

(21) Appl. No.: 10/801,049

(22) Filed: Mar. 15, 2004

(30) Foreign Application Priority Data
Mar. 17, 2003 (DE) 10311561.7

(51) Int. Cl. C07D 333/02; C08K 5/45

(52) U.S. Cl. 524/84; 549/29

(57) ABSTRACT

3,4-Alkylenedioxythiophene derivatives of the formulae I and II

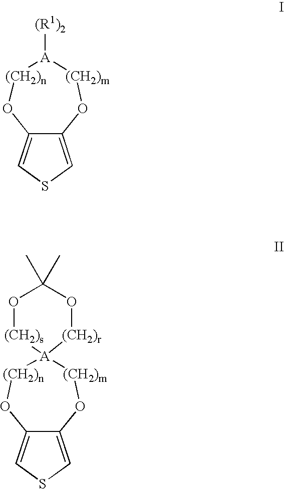

where n and m are each an integer, their preparation and their use for preparing electrically conductive oligomers or polymers, in particular for producing solid electrolytes for electrolytic capacitors, and also oligomers or polymers in which these 3,4-alkylenedioxythiophene derivatives are present as repeating units.

The invention relates to 3,4-alkylenedioxythiophene derivatives, a process for preparing them and their use for preparing electrically conductive oligomers or polymers, in particular for producing solid electrolytes for electrolytic capacitors. Furthermore, the invention relates to oligomers or polymers in which these compounds are present as repeater units.

Organic conductive polymers have a wide range of uses. Examples which may be mentioned are their use for producing polymer batteries, diodes or transistors or solar cells. Organic conductive polymers employed are, for example, systems based on polyacetylene, poly(p-phenylene), polythiophene or polypryrrol. EP 340 512 B 1 discloses the use of organic conductive polymers as solid electrolytes for electrolytic capacitors.

Some electrically conductive oligomers or polymers prepared from thiophene derivatives are known. A particular example is poly[3,4-ethylenedioxythiophene] (PEDT), which is used, in particular, in the cationic form with polystyrenesulphonic acid (PSS) as anionic component. PEDT-PSS is commercially available under the trade name Baytron® P.

EP 340 512 B1 describes the preparation of a solid electrolyte from 3,4-ethylene-1,2-dioxy-thiophene and the use of its cationic polymer (PEDT) prepared by oxidative polymerization as solid electrolyte in electrolytic capacitors. PEDT as replacement for manganese dioxide or for charge transfer complexes in solid electrolyte capacitors reduces the equivalent series resistance of the capacitor as a result of the increased electric conductivity and improves the frequency behaviour.

The leakage current of such a capacitor depends essentially on the quality of the polymer film: if graphite or silver permeates through the polymer film and thus comes into contact with the dielectric, the leakage current increases drastically since defects in the oxide layer can no longer be encapsulated by the local destruction of the conductive polymer (self-healing effect).

After a chemical polymerization, the salts, i.e. excess oxidant and also its reduced form, may have to be washed out to achieve layers of appropriate quality. Otherwise, crystallization of salts can over the course of time lead to an increased series resistance due to the occurrence of contact resistances. In addition, the crystals can damage the dielectric or the outer contact layers when the capacitor is mechanically stressed, so that the leakage current increases. It is therefore desirable to suppress the crystallization of salts of the oxidant and residual salts of its reduced form which remain in the capacitor despite washing.

There is therefore a continuing need to increase the conductivity and quality as is known for layers of poly(3,4-ethylenedioxythiophene), especially with a view to the above-described use in electrolytic capacitors, in order to achieve higher performance. It is therefore particularly desirable to achieve further decreases in the equivalent series resistance and the leakage current of solid electrolyte capacitors. In addition, simple production of the layers or the electrolytic capacitors is desirable.

To be able to match the polymer properties, in particular the solubility and conductivity of the polymer, to the respective requirements in a targeted way, it is necessary to have a large number of suitable monomer building blocks available.

It is an object of the invention to provide novel thiophene derivatives and find ways of preparing them. In particular, thiophene derivatives which are suitable as solid electrolytes in electrolytic capacitors are to be made available, and conductivity and quality, in particular in respect of better binding of residual salts and homogeneity, are to be improved in comparison with known polymers, for example poly(3,4-ethylenedioxythiophene).

Novel 3,4-alkylenedioxythiophene derivatives which have two hydroxyalkyl or hydroxy groups in the cyclic alkylene unit have now been able to be prepared. These compounds have the characteristics that, inter alia, the hydroxyalkyl or hydroxy groups increase the solubility in polar solvents and the compounds can be modified further by means of further reactions on the hydroxyalkyl or hydroxy groups. The electronic structure can in this way be influenced in a targeted manner, which makes the compounds particularly interesting as monomers for preparing conductive polymers. Furthermore, the presence of the hydroxyalkyl or hydroxy groups increases the interaction with capacitor materials.

The invention accordingly provides compounds of the formula I

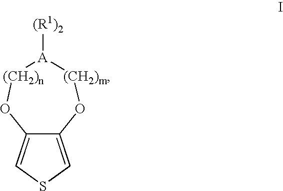

where

n and m are each, independently of one another, an integer from 1 to 5,

A is a methylene or ethylene radical,

where A bears two substituents R¹ and

R¹ is, in each case, a C₁-C₆-hydroxyalkyl radical, preferably a hydroxymethyl or hydroxyethyl radical, particularly preferably a hydroxymethyl radical or a hydroxyl radical.

The two substituents R¹ may be identical or different, preferably identical. An ethylene radical may bear the two substituents R¹ on the same C atom or on different C atoms, preferably on different C atoms.

Preferred compounds according to the invention are those of the formula I-a

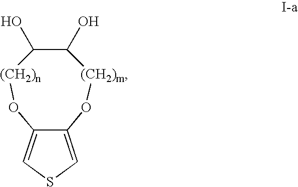

where n and m are each, independently of one another, an integer from 1 to 5.

Further preferred compounds according to the invention are those of the formula I-b

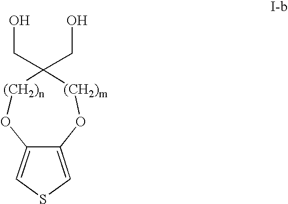

Where n and m are each, independently of one another, an integer from 1 to 5.

Preference is given to n and m each being, independently of one another, an integer from 1 to 3; n and m are particularly preferably the same number and are very particularly preferably each 1.

In order to carry out further reactions, it can be advantageous for the hydroxyalkyl or hydroxy groups to be protected. A known way of introducing a protective group for vicinal diols is reaction with acetone to form 1,3-dioxolanes. The protective group is stable to bases and can be removed again by treatment with acids. Even pairs of hydroxy groups which are not vicinal, e.g. those in compounds of the formula I and I-b, can be protected by reaction with acetone.

The invention therefore further provides compounds of the formula II

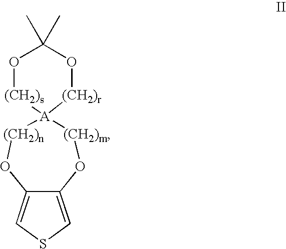

where r and s are each, independently of one another, 0 or an integer from 1 to 6 and n, m and A are as defined above.

Preference is given to r and s each being, independently of one another, 0 or 1; r and s are particularly preferably the same number and are very particularly preferably each 0 or 1.

The invention preferably provides compounds of the formula II-a

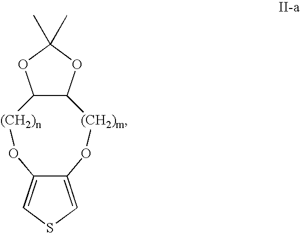

where n and m are each, independently of one another, an integer from 1 to 5.

The invention also preferably provides compounds of the formula II-b

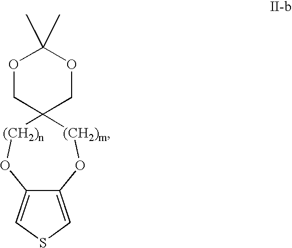

where n and m are each, independently of one another, an integer from 1 to 5.

Preference is given to n and m each being, independently of one another, an integer from 1 to 3; n and m are particularly preferably the same number and are very particularly preferably each 1.

Compounds of the formulae I and II can be prepared, for example, by reacting suitable thiophenes with α,Ω-tosylate-substituted alkylenes; for the preparation of compounds of the formula II, the protective group for vicinal diols is subsequently reintroduced if necessary, for example by reaction with acetone. Compounds of the formula I here and below also include the preferred compounds of the formula I-a and I-b; compounds of the formula II here and below also include the preferred compounds of the formula II-a and II-b.

The invention therefore also provides a process for preparing a compound of the formula I or II, in which a thiophene of the formula III

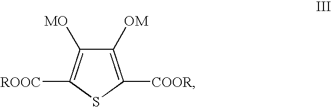

where

R is C₁-C₁₈-alkyl and

M is H, Li, Na or K,

is reacted with a compound of the formula IV

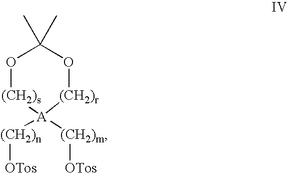

where n, m, r, s and A are as defined above and Tos is p-toluenesulphonyl, to form a compound of the formula V

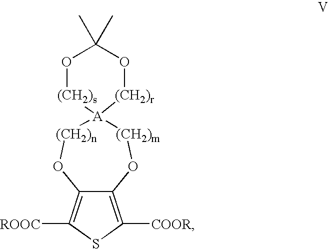

where

R, A, r, s, n and m are as defined above,

and the compound of the formula V is subsequently hydrolyzed, acidified and decarboxylated.

The process of the invention is preferably one in which a thiophene of the formula III is reacted with a compound of the formula IV-a

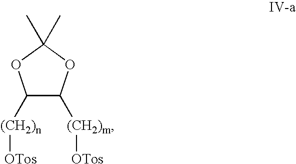

where

n and m are each, independently of one another, an integer from 1 to 5 and Tos is p-toluenesulphonyl,

to form a compound of the formula V-a

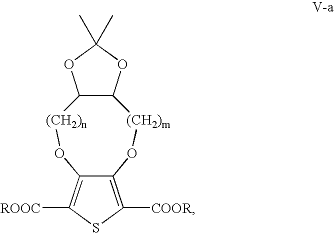

where

R, n and m are as defined above,

and the compound of the formula V-a is subsequently hydrolyzed, acidified and decarboxylated.

The process of the invention is furthermore preferably one in which a thiophene of the formula III is reacted with a compound of the formula IV-b

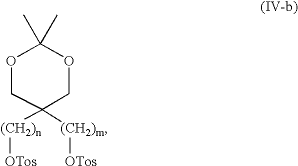

where

n and m are each, independently of one another, an integer from 1 to 5 and Tos is p-toluenesulphonyl,

to form a compound of the formula V-b

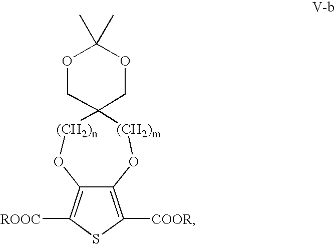

where

R, n and m are as defined above,

and the compound of the formula V-b is subsequently hydrolyzed, acidified and decarboxylated.

R in the formulae III and V is preferably C₁-C₆-alkyl, for example ethyl or methyl, particularly preferably methyl. Compounds of the formula V here and below also include the preferred compounds of the formulae V-a and V-b.

The reaction of the thiophene of the formula III with the compound of the formula IV can, for example, be carried out at atmospheric pressure under a protective gas atmosphere (Ar, N₂) in dipolar, aprotic solvents in the presence of a base such as potassium carbonate. Compounds of the formula IV here and below also include the preferred compounds of the formulae IV-a and IV-b.

Suitable solvents are, for example, N-methyl-2-pyrrolidone (NMP), dimethylformamide, dimethylacetamide, dimethyl sulphoxide or high-boiling ketones. Preference is given to using N-methyl-2-pyrrolidone as solvent.

The reaction can be carried out, for example, at a temperature of from 80 to 160° C., preferably from 90 to 120° C.

Acidification can be carried out, for example, by addition of acids, in particular acetic acid, at temperatures of from 10 to 50° C. Preference is given to an acid in such an amount that a pH of from 1 to 5 is established at the temperature of the reaction. In this way, the protective group for the dihydroxy grouping can be removed, preferably in a tailored manner.

The hydrolysis can be carried out under generally customary conditions for such a reaction. For example, the compound of the formula V can be heated in dilute sodium or potassium hydroxide solution and subsequently be neutralized with hydrochloric or sulphuric acid. Such a procedure is known, for example, from U.S. Pat. No. 5,111,327. The neutralization with hydrochloric or sulphuric acid enables, preferably in a tailored manner, the ester groups to be removed and the free dicarboxylic acid to be obtained.

The decarboxylation can also be carried out in a manner known per se (U.S. Pat. No. 5,111,327 and EP 339 340 B1). For example, the compound of the formula V after hydrolysis and acidification is heated in ethanolamine to high temperatures, e.g. from 160 to 200° C., or in a dipolar aprotic solvent such as dimethylacetamide or dimethyl sulphoxide in the presence of a catalyst such as basic copper carbonate or copper chromite/quinoline.

If desired, the protective group for vicinal diols can subsequently be reintroduced by known methods, e.g. by reaction with acetone to form 1,3-dioxolanes.

Reaction of the compounds of the formula III, where M and R are as defined above, with compounds of the formula IV, where r, s, A, m, n and Tos are as defined above, firstly gives, in a tailored manner, the compounds of the formula V, where r, s, A, n, m and R are as defined above. The reaction can be carried out under the above-described conditions. The targeted preparation of the compounds of the formula V can be advantageous in that a prepurification can be carried out. In this way, the compounds of the formula I can be obtained in a particularly high purity in preferred embodiments by subsequent hydrolysis, acidification and decarboxylation. Furthermore, the compounds of the formula V may have a better storage stability than the compounds of the formula I.

The compounds of the formula I and II can be used for preparing electrically conductive oligomers and polymers. Here, it is possible to use either only one compound of the formula I or II as monomer or a mixture of various compounds which come under the definition of the formulae I and II. Furthermore, it is also possible to add further thiophene derivatives, in particular 3,4-ethylenedioxythiophene, which are commercially available under the trade name Bayton®M, as monomers in addition to one or more compounds of the formulae I and II.

The polymerization is carried out in a manner corresponding to the polymerization of known thiophene derivatives. It can, for example, be carried out oxidatively using oxidants such as iron(III) chloride or other iron(III) salts, H₂O₂, sodium or potassium peroxodisulphate, potassium dichromate, potassium permanganate, or electrochemically.

The invention therefore also provides for the use of compounds of the formulae I and II for preparing electrically conductive oligomers and polymers and provides electrically conductive oligomers and polymers which are prepared by polymerization of a compound of the formula I and/or II.

In particular, the invention provides electrically conductive oligomers and polymers comprising, as repeating units, structural units of the formula VI

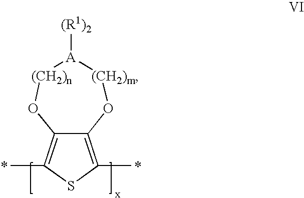

where

A and R¹ are as defined above and

n and m are each, independently of one another, an integer from 1 to 5 and x is an integer from 2 to 10 000,

and/or the formula VII,

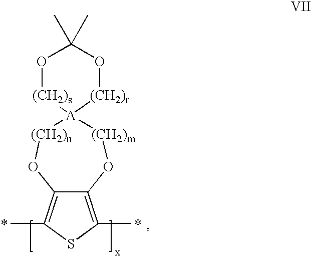

where

n, m, r, s and A are as defined above for the formula II and x is an integer from 2 to 10 000.

Preferred electrically conductive oligomers and polymers according to the invention are ones which comprise structural units of the formula VI-a and/or VII-a

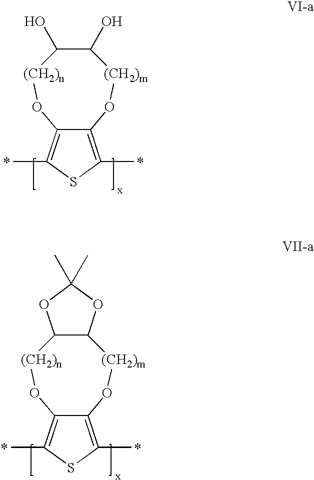

where n, m and x are as defined above, as repeating units.

Further preferred electrically conductive oligomers and polymers according to the invention are ones which comprise structural units of the formula VI-b and/or VII-b

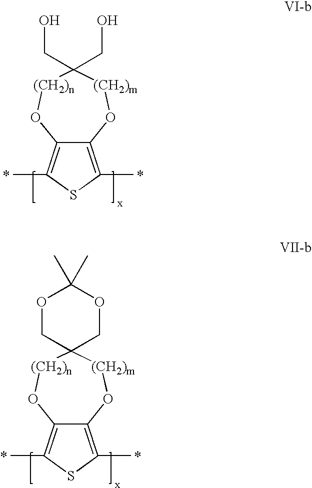

where n, m and x are as defined above, as repeating units.

x is preferably from 2 to 5 000, particularly preferably from 2 to 100.

The structural units of the formulae VI,VI-a, VI-b and VII, VII-a, VII-b can, as shown in the formulae, be uncharged. However, it is also possible for them to bear a positive charge. In this case, the polymers of the invention contain anions as counterions. These anions in turn preferably have a polymeric structure; a particularly preferred polyanion is polystyrenesulphonate.

The oligomers or polymers of the invention can, for example, be used as hole injection layer in organic light-emitting diodes, as smoothing layer for ITO layers in organic light-emitting diodes, as conductive layers in inorganic light-emitting diodes, as colour-imparting electrochromic or ion-storing counterelectrode in electrochromic assemblies, for elimination of static electricity, for plated-through holes in printed circuits, in corrosion protection, in sensors or in organic field effect transistors.

The invention also provides mixtures of the novel thiophene derivatives of the formula I or II with 3,4-ethylenedioxythiophene (EDT).

The following examples are not intended to restrict the scope of the present invention but serve exclusively to illustrate the invention.

### EXAMPLES

### Example 1

2.46 g (0.0106 mol) of dimethyl 3,4-dihydroxythiophene-1,2-dicarboxylate (Bayer AG, Leverkusen) and 5 g (0.0106 mol) of 1,4-di-o-tosyl-2,3-isopropylidene-D-threitol (Aldrich) were stirred together with 3.66 g (0.0265 mol) of potassium carbonate in 130 ml of dried N-methylpyrrolidone (NMP) at 100° C. for 18 hours. To work up the reaction mixture, it was admixed with water and methylene chloride and shaken until neutral. The organic phase was separated off and dried over sodium sulphate. Evaporation of the organic phase and drying gave 2.2 g of a brown crude product which contained the desired product having a molar mass of 358 as main component (analysis via GC-MS).

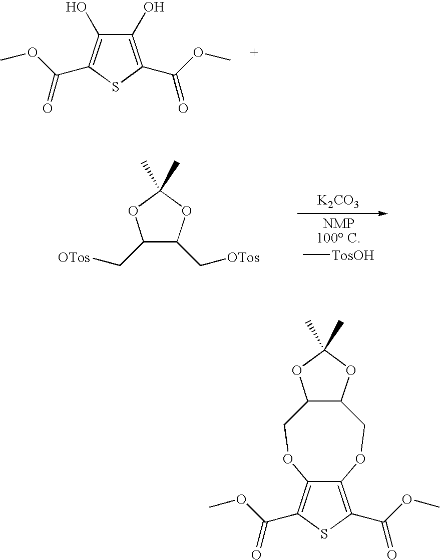

### Example 2

Chromatography on silicon dioxide using a mixture of toluene/ethyl acetate (firstly 10 parts by volume of toluene and 1 part by volume of ethyl acetate, then 5:1, 3:1 and finally 1:1) as eluant gave, after elimination of the protective group, the desired diol having a molar mass of 318 (analysis by GC-MS after conversion into the trimethylsilyl derivative (TMS derivative); di-TMS MW 462).

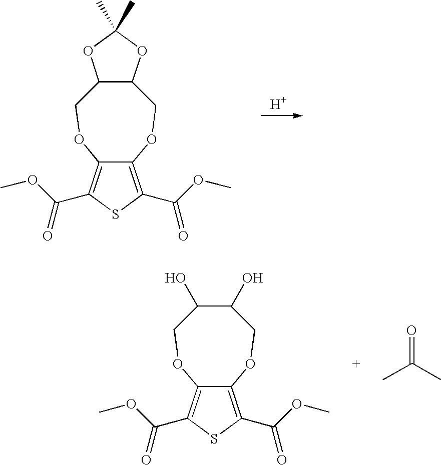

### Example 3

Example 1 was repeated with the amount of starting materials being increased by a factor of four. This gave 9.3 g of a dark brown crude product which, after purification by chromatography and cleavage in a manner analogous to Example 2, gave 2.0 g (0.0063 mol, 14.9% of theory) of a yellow crystalline solid as product. The free thiophene compound can be set free in a known manner by ester cleavage (hydrolysis and acidification) and decarboxylation, for example using a method analogous to that described in U.S. Pat. No. 5,111,327 or EP 339 340 B1.

### Example 4

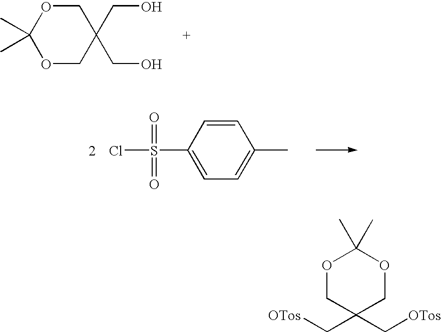

10 g (56.8 mmol) of monochetalized pentaerythritol together with 24.2 g (150 mmol) of triethylamine are placed in a reaction vessel. At room temperature, 22.9 g (120 mmol) of p-toluenesulphonyl chloride in 50 ml of methylene chloride are added dropwise to this solution. A slightly exothermic reaction occurs with the solution becoming turbid. The mixture is stirred further for about 3.5 hours at room temperature and subsequently for 3 hours at 40-45° C.

The entire mixture is evaporated to dryness on a rotary evaporator and the residue is washed three times with 200 ml of methanol. The methanolic solution is decanted off and the white residue is dried in a water pump vacuum.

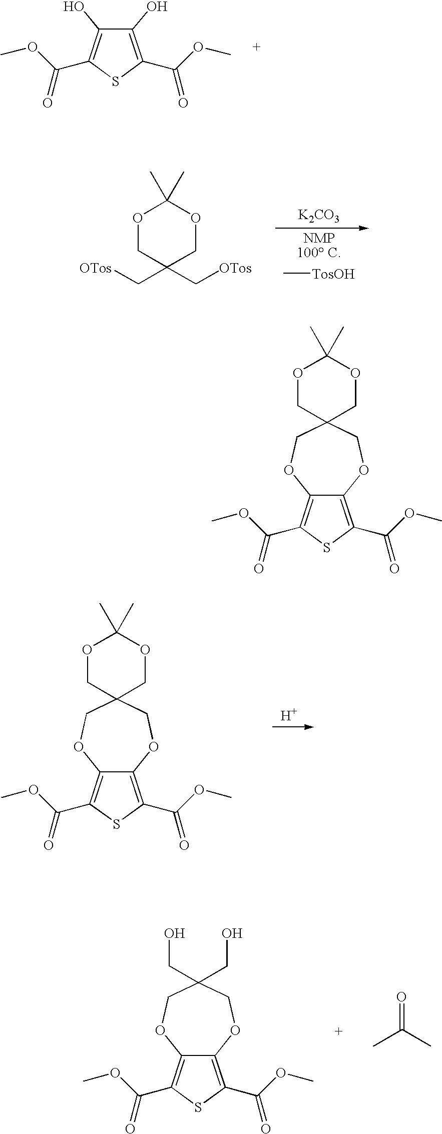

2.55 g (0.011 mol) of dimethyl 3,4-dihydroxythiophen-1,2-dicarboxylate and 5 g (0.011 mol) of the ditosylate of monoprotected pentaerythritol are stirred with 3.8 g (0.028 mol) of potassium carbonate in 100 ml of N-methylpyrrolidone at 100° C. for 18 hours. To work up the reaction mixture, it is admixed with water and methylene chloride and washed until neutral. The organic phase is separated off and dried over sodium sulphate. Evaporation of the organic phase and drying gave 2.8 g of product.

Analysis: GC-MS shows the desired molecule having a molar mass of 372 as main product.

Further purification is carried out by chromatography on silicon dioxide in a manner analogous to Example 2. The protective group is removed in a manner analogous to Example 3. This gives the desired product (analysis by GC-MS after conversion into the trimethylsilyl derivative (TMS derivative);di-TMS MW 476).

1. Compound of the formula I 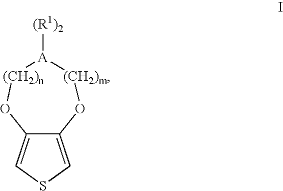
where
n and m are each, independently of one another, an integer from 1 to 5,
A is a methylene or ethylene radical,
where A bears two substituents R¹ and
R¹ is, in each case, a C₁-C₆-hydroxyalkyl radical, preferably a hydroxymethyl or hydroxyethyl radical, particularly preferably a hydroxymethyl radical or a hydroxyl radical.

2. Compound according to claim 1 having the formula I-a 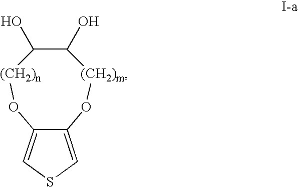
where
n and m are each, independently of one another, an integer from 1 to 5.

3. Compound according to claim 1 having the formula I-b 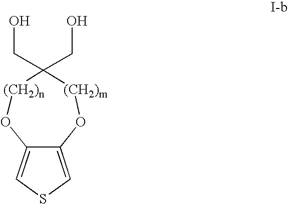
where n and m are each, independently of one another, an integer from 1 to 5.

4. Compound according to claim 1, characterized in that n and m are each 1.

5. Compound of the formula II 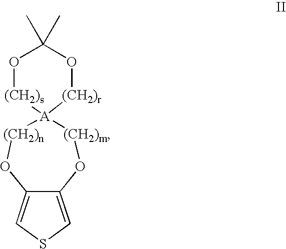
where r and s are each, independently of one another, an integer from 1 to 6,
n and m are each, independently of one another, an integer from 1 to 5,
A is a methylene or ethylene radical.

6. Compound according to claim 5 of the formula II-a 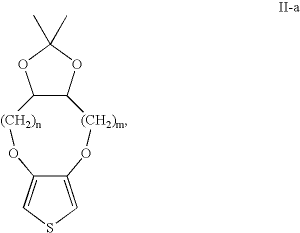
where
n and m are each, independently of one another, an integer from 1 to 5.

7. Compound according to claim 5 having the formula II-b 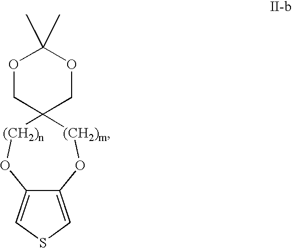
where n and m are each, independently of one another, an integer from 1 to 5.

8. Compound according to claim 5, characterized in that n and m are each 1.

9. Process for preparing a compound according to claim 1, characterized in that a thiophene of the formula III 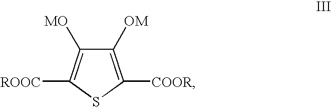
where
R is C₁-C₁₈-alkyl and
M is H, Li, Na or K,
is reacted with a compound of the formula IV 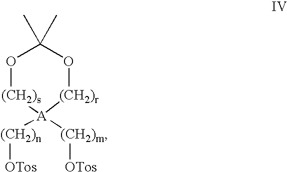
where
r and s are each, independently of one another, 0 or an integer from 1 to 6,
n and m are each, independently of one another, an integer from 1 to 5,
A is a methylene or ethylene radical,
Tos is p-toluenesulphonyl,
to form a compound of the formula V 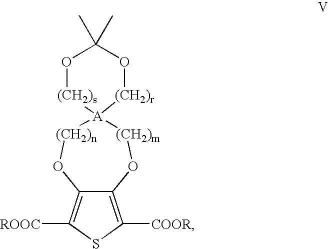
where A, R, r, s, n and m are as defined above for the formulae III and IV,
and the compound of the formula V is subsequently hydrolyzed, acidified and decarboxylated.

10. Process according to claim 9, characterized in that a thiophene of the formula III 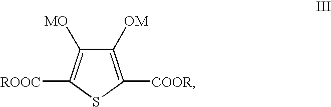
where M and R are as defined in claim 9,
is reacted with a compound of the formula IV-a 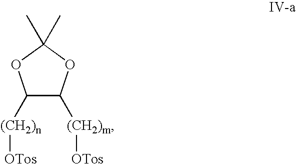
where
n and m are each, independently of one another, an integer from 1 to 5 and
Tos is p-toluenesulphonyl,
to form a compound of the formula V-a 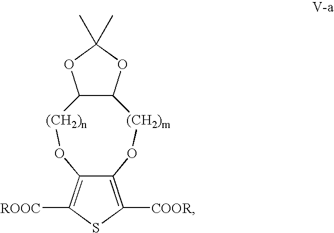
where
R, n and m are as defined for the formulae III and IV-a,
and the compound of the formula V-a is subsequently hydrolyzed, acidified and decarboxylated.

11. Process according to claim 9, characterized in that a thiophene of the formula III 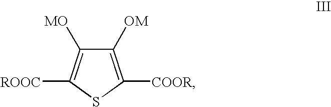
where M and R are as defined in claim 9,
is reacted with a compound of the formula IV-b 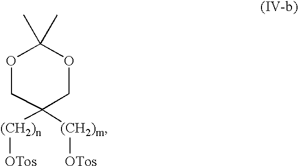
where
n and m are each, independently of one another, an integer from 1 to 5 and
Tos is p-toluenesulphonyl,
to form a compound of the formula V-b 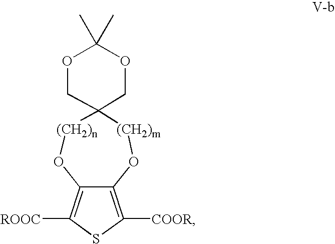
where
R, n and m are as defined for the formulae III and IV-b,
and the compound of the formula V-b is subsequently hydrolyzed, acidified and decarboxylated.

12. Use of a compound according to claim 1 for preparing an electrically conductive oligomer or polymer.

13. Use of a compound according to claim 1 in the production of capacitors.

14. Use of a mixture comprising at least one compound according to claim 1 and 3,4-ethylenedioxythiophene in the production of capacitors.

15. Electrically conductive oligomer or polymer comprising structural units of the formula VI 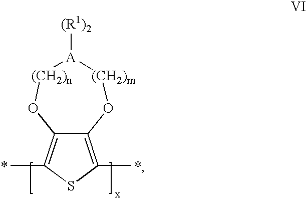
where
A is a methylene or ethylene radical,
where A bears two substituents R¹ and
R¹ is, in each case, a C₁-C₆-hydroxyalkyl radical, preferably a hydroxymethyl or hydroxyethyl radical, particularly preferably a hydroxymethyl radical, or a hydroxyl radical,
n and m are each, independently of one another, an integer from 1 to 5 and
x is an integer from 2 to 10 000,
and/or the formula VII, 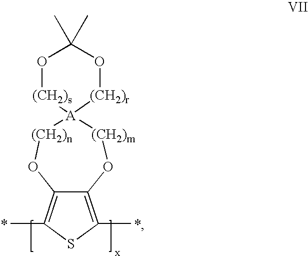
where
r and s are each, independently of one another, 0 or an integer from 1 to 6
and n, m, A and x are as defined for the formula VI.

16. Electrically conductive oligomer or polymer according to claim 15 comprising structural units of the formula VI-a 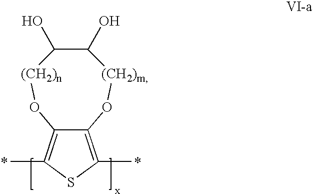
where n, in and x are as defined in claim 15, and/or of the formula VII-a, 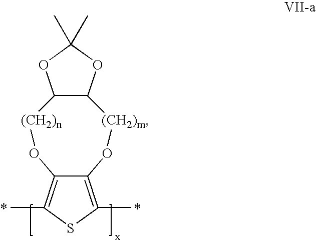
where n, m and x are as defined in claim 15.

17. Electrically conductive oligomer or polymer according to claim 15 comprising structural units of the formula VI-b 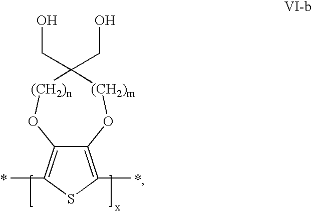
where n, m and x are as defined in claim 15, and/or of the formula VII-b 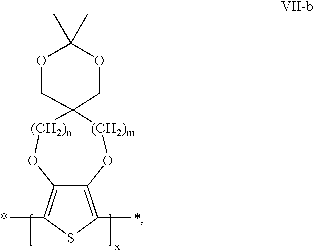
where n, m and x are as defined in claim 15.

18. Electrically conductive oligomer or polymer according to claim 15, characterized in that it is a copolymer comprising structural units of the formula VI and/or VII together with 3,4-ethylenedioxythiophene structural units.

19. Use of an electrically conductive oligomer or polymer according to claim 15 as hole injection layer in organic light-emitting diodes, as smoothing layer for ITO layers in organic light-emitting diodes or as colour-imparting electrochromic or ion-storing counterelectrode in electrochromic assemblies.

20. Use of an electrically conductive oligomer or polymer according to claim 15 in organic field effect transistors.

21. Use of an electrically conductive oligomer or polymer according to claim 15 in elimination of static electricity, in through-plated holes of printed circuits, in corrosion protection, in sensors or as conductive layer in inorganic light-emitting diodes.

22. Compound of the formula V 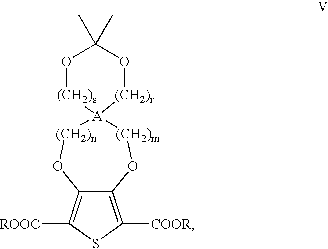
where
R is C₁-C₁₈-alkyl,
r and s are each, independently of one another, 0 or an integer from 1 to 6,
n and m are each, independently of one another, an integer from 1 to 5,
A is a methylene or ethylene radical.

23. Compound according to claim 22 having the formula V-a or V-b 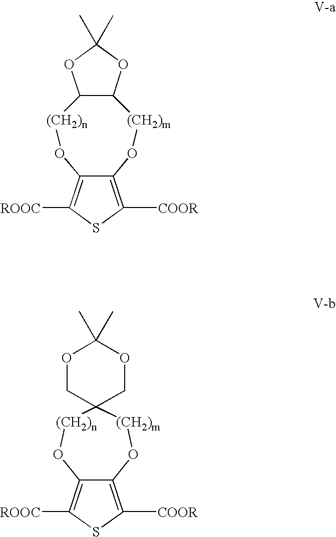
where R, n and m are as defined in claim 22.
